(19) United States
(12) Patent Application Publication
(10) Pub. No.: US 2021/0129152 A1
(43) Pub. Date: May 6, 2021

(54) Connector for Flexible Tubing

(71) Applicant: Global Life Sciences Solutions USA LLC, Marlborough, MA (US)

(72) Inventors: Yasser Ali, Marlborough, MA (US); Philip Vanek, Marlborough, MA (US)

(21) Appl. No.: 17/061,958

(22) Filed: Oct. 2, 2020

Related U.S. Application Data
(60) Provisional application No. 62/930,060, filed on Nov. 4, 2019.

(51) Int. Cl. B01L 3/00; C12M 1/00; C12M 1/12; B01L 9/00

(52) U.S. Cl. CPC B01L 3/563; C12M 27/18; C12M 37/04; B01L 3/561; B01L 2300/0832; B01L 9/50; B01L 2200/026; B01L 2200/0689; B01L 2300/0609; B01L 9/00

(57) ABSTRACT

A connector for flexible tubing comprises a first hollow insert for allowing fluid flow, having a proximal end configured to fit inside the tubing, a first outer part, a joining member rigidly connecting the first insert to the outer part at a region spaced from the proximal end of the first insert and a generally annular gap between the first insert and the first outer part, which forms a tubing accepting opening at or adjacent the proximal end of the insert, the first outer part further comprising plural resilient protrusions extending into the gap, allowing the tubing to be inserted, but resisting removal of the tubing once inserted and wherein at least the insert, the joining member and the outer part are formed as a single integral molding.

### CROSS REFERENCE TO RELATED APPLICATIONS

This application claims the priority benefit of U.S. Provisional Application No. 62/930,060 filed Nov. 4, 2019, which application is incorporated herein by reference.

### TECHNICAL FIELD

The present invention generally relates to a connector for making connection with flexible tubing. More specifically the present invention relates to a connector which makes leak-proof connection with one or more flexible tubing used in the field of biotechnology.

### BACKGROUND

There are several types of connectors that are used for making connection between various types of tubing. A commonly seen connector with a simple design is one used for joining two pieces of garden hose. Other areas where connectors are used commonly are food industry and plumbing, but these connectors typically have a complex design and are not designed to suit the needs of a laboratory environment. With rise in biotechnological research, the demand for lab equipment which is easy to manufacture, use, clean and sterilize has also gone up. Connectors which can be used for the purpose of joining flexible tubing or making connection with another lab equipment are therefore required to meet the above features.

One problem with the connectors disclosed in the prior art is that those connectors are prone to causing fluid leakage or forming dead legs, both of which are undesirable, especially when dealing with manufacturing of biopharmaceuticals or research in the area. Fluid leakage not only causes reduced final output due to loss of cells or biomolecules in the fluid leaked, it can also cause contamination. Formation of dead legs further increases the chances atrophication and of non-uniform cell growth as the spent culture media gets trapped without getting displaced by fresh media.

One type of connector is disclosed in U.S. Pat. No. 7,100,947B2, which describes a tubing and connector system including a plastic tubing with a helical or spiral outer surface, a lock nut and a connector fitting, wherein the lock nut engages the outer surface of the tubing to squeeze the tubing between the lock nut and the fitting. The problem with such a connector is that the connector fitting and the lock nut need to be manufactured as separate parts and the lock nut needs to be manufactured with different thread pitches to engage over tubes which differ in their outer corrugation. Further, most of the tubing used in biomanufacturing is plain tubing without any corrugations, so this type of connector would not be suitable for the intended purpose.

Another type of connector is disclosed in U.S. Pat. No. 9,631,754B2, where a clamp can be used to secure the connection between a flexible tube and a tubing connector. This again requires two separate pieces to be manufactured i.e. the connector and the clamp. Also, this patent mentions that re-tightening of the clamp may be needed due to creep deformation that occurs in some malleable tubing materials that can result in a reduction in the elastic interference created on the connector fitting. This can eventually result in a weaker tubing to fitting joint. Under increased fluid pressure, a weaker radial compression force on the fitting can result in dissociation of the tubing and fitting resulting in a fluid leak.

Yet another type of connector is disclosed in U.S. Pat. No. 7,527,300B2, where the connector comprises a fitting and a collar. The fitting is adapted to receive the flexible tubing thereon and includes an exteriorly disposed barb for engaging the interior walls of the flexible tubing. The collar is adapted to engage the fitting at least about the neck and barb and includes an interiorly disposed flange portion having a contour that is shaped to cooperate with the surfaces of the barb to drive the flexible tubing over the barb and the neck as the collar and fitting are assembled with one another. Like the above prior art, this patent also discloses a two-part connector system. Also, the threading on the clamp engages the tube when twisted resulting in a friction fit not a mechanical locking and thus prone to dissociation of the tubing and fitting.

Thus, there is a need for an improved connector that can provide a leak-proof connection between a flexible tubing and a connector fitting, and that can be easily manufactured at low cost by simple processes like one-piece molding or casting, thus making it suitable for use in biotechnology and related fields.

### SUMMARY OF THE INVENTION

An object of the embodiments of the present invention is to provide an improved connector for flexible tubing and the method to form a connection using the connector.

One advantage of the invention is that the connector prevents leakage of fluids at the connection joint.

Another advantage of the invention is that the connector prevents forming of dead legs in and around the connection joint.

Another advantage of the invention is that the connector can be manufactured easily and at low cost by single integral molding.

Yet another advantage of the invention is that the connector can be conveniently and effectively sterilized as it is a single piece with no joints.

In aspects of the invention, a connector for flexible tubing comprises a first hollow insert for allowing fluid flow, having a proximal end configured to fit inside the tubing, a first outer part, a joining member rigidly connecting the first insert to the outer part at a region spaced from the proximal end of the first insert and a generally annular gap between the first insert and the first outer part, which forms a tubing accepting opening at or adjacent the proximal end of the insert, the first outer part further comprising plural resilient protrusions extending into the gap, allowing the tubing to be inserted, but resisting removal of the tubing once inserted and wherein at least the insert, the joining member and the outer part are formed as a single integral molding.

In other aspects of the invention, the single integral molding is made of plastics or metal material.

In other aspects of the invention, the resilient protrusions are formed as an element separate from and insertable into the annular gap.

In other aspects of the invention, the hollow insert is configured to make a seal between an internal face of the tubing and an outer diametral face of the insert.

In other aspects of the invention, the connector further includes a second insert and a second outer part fluidically connected to the first insert and the first outer part to provide a further tube accepting opening, the second insert and the second outer part being rigidly connected also by the joining member, the connector thereby being configured to accept tubing at more than one fluidically connected openings and to allow fluid to flow through the first insert and into the second insert.

In other aspects of the invention, the tubing has one or more ends formed with protrusions complimentary to the protrusions of the first and/or second outer parts of the connector further configured to allow the tubing to be inserted over the connector but resist removal of the tubing once inserted.

In other aspects of the invention, the joining member forms part of any one of a bioreactor bag, a disposable fluid containment, a fluid manifold and a bioprocessing instrument.

In other aspects of the invention, the protrusions of the first and/or second outer part, and the protrusions formed on the tube, together are in the form of ratcheting teeth.

In other aspects of the invention, the protrusions of the first and/or the second outer part, are inclined away from the opening, or have ends which are so inclined.

More advantages and benefits of the present invention will become readily apparent to the person skilled in the art in view of the detailed description below.

### DRAWINGS

The invention will now be described in more detail with reference to the appended drawings, wherein:

FIG. 1 shows a longitudinal sectional view of an exemplary connector 20 of the invention;

FIG. 2 shows an end view of the exemplary connector 20 as illustrated in FIG. 1;

FIG. 3 shows a longitudinal sectional view of an exemplary connector 100 of the invention that is connected to a flexible tubing;

FIG. 4 shows an end view of the exemplary connector 100 as illustrated in FIG. 3;

FIG. 5 shows a longitudinal sectional view of a two-way connector 200 according to a first embodiment of the invention;

FIG. 6 shows a longitudinal sectional view of a connector 300 according to a second embodiment of the invention.

FIG. 7 shows a longitudinal sectional view of a connector 400 according to a third embodiment of the invention.

### DETAILED DESCRIPTION

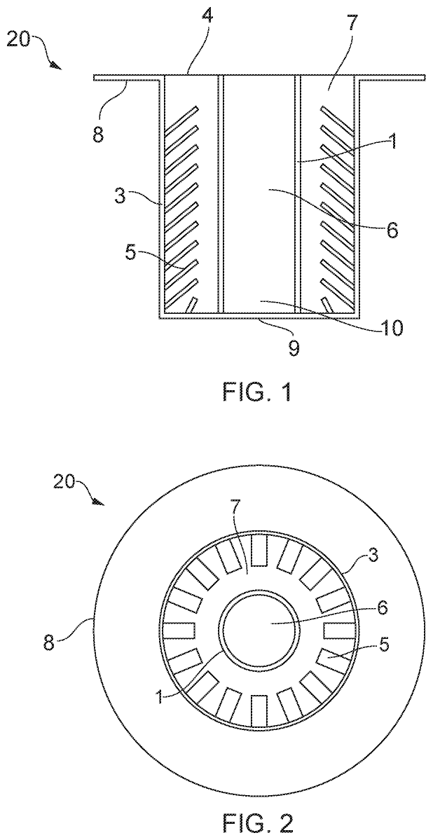

FIG. 1 shows a longitudinal sectional view of an exemplary connector 20 of the invention. The connector 20 comprises a hollow insert 1 enclosing a passage 6. The passage 6 allows fluid flow through the insert 1. Proximal end 10 of the insert 1 is configured to fit inside a tubing. In the exemplary connector 20, the proximal end 10 of the insert 1 is of a blunt design. In other examples, the proximal end 10 could be of a tapered design to facilitate insertion inside a tubing. The insert 1 is configured to make a seal between an internal face of the tubing and an outer diametral face of the insert 1. This can be achieved by keeping the largest outside diameter of the insert 1 equal to or more than inside diameter of the tubing. The insert 1 is surrounded by an outer part 3 such that there is a generally annular gap 7 between the insert 1 and the outer part 3. The annular gap 7 forms a tubing accepting opening 9 at or adjacent to the proximal end 10 of the insert 1. As shown in FIG. 1, the exemplary connector 20, the outer part 3 and the insert 1 are of cylindrical shapes with the insert 1 placed concentrically inside the outer part 3. In another example, a connector could be of a different shape to be able to fit inside differently shaped tubing. A joining member 4 rigidly connects the insert 1 to the outer part 3 at a region spaced from the proximal end 10 of the insert 1. This can be achieved by manufacturing the connector 20 as a single integral molding made of plastics, metal or any other material. The outer part 3 comprises a plurality of resilient protrusions 5 extending into the annular gap 7. The protrusions 5 are configured to allow the tubing to be inserted into the tubing accepting opening 9 but resist removal of the tubing once inserted. In the exemplary connector 20, the protrusions 5 are inclined away from the opening 9 such that once the tubing is pushed inside the opening 9 into the annular gap 7, the tubing cannot be pulled out. In another example, the protrusions 5 could be in the form of ratcheting teeth. The connector also comprises an optional flange 8 to facilitate attachment of the connector 20 to a bioreactor, a cell bag, a manifold or other biotechnological equipment.

FIG. 2 shows an end view of the exemplary connector 20 as illustrated in FIG. 1. The connector 20 comprises the hollow insert 1 enclosing the passage 6 for fluid flow. The exemplary connector 20, the insert 1 and the outer part 3 have a circular cross section and are thus suitable to connect to a cylindrical tubing. In other examples, an insert and/or an outer part of a connector could have a different geometry to be able to fit inside differently shaped tubing. The outer portion 3 comprises the protrusions 5 extending into the annular gap 7 from the outer part 3.

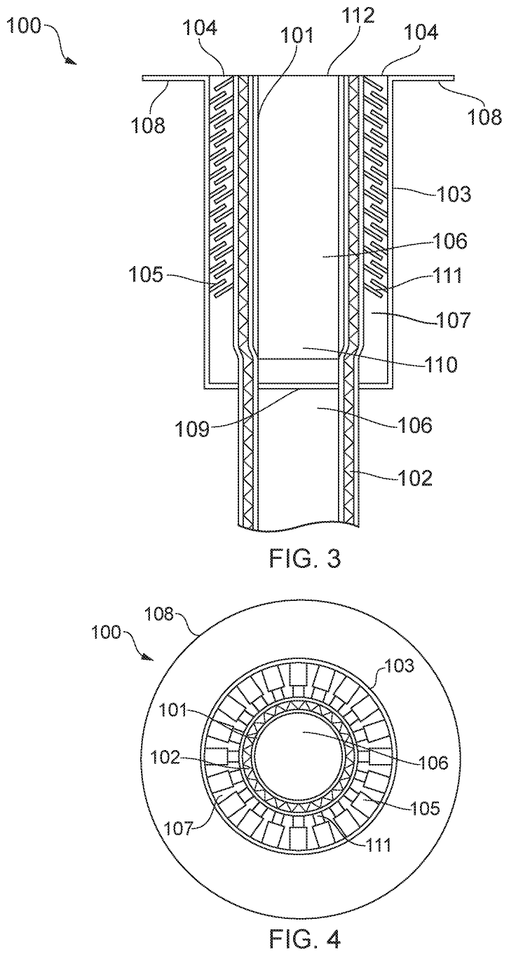

FIG. 3 shows a longitudinal sectional view of an exemplary connector 100 of the invention that is connected to a flexible tubing 102. The connector 100 comprises a hollow insert 101 enclosing a passage 106. The passage 106 allows fluid flow through the insert 101. Proximal end 110 of the insert 101 is configured to fit inside the tubing 102. In the exemplary connector 100, the proximal end 110 of the insert 101 has a tapered shape to facilitate entry of the insert 101 inside the tubing 102. In other examples the proximal end 110 could be of a different geometry for example, a blunt end or an end with barbs. The insert 101 is surrounded by an outer part 103 such that there is a generally annular gap 107 between the insert 101 and the outer part 103. The annular gap 107 forms a tubing accepting opening 109 at or adjacent to the proximal end 110 of the insert 101. A joining member 104 rigidly connects the insert 101 to the outer part 103 at a region spaced from the proximal end 110 of the insert 101. The outer part 103 comprises a plurality of resilient protrusions 105 extending into the annular gap 107. The tubing 102 also comprises a plurality of protrusions 111 that are complimentary to the protrusions 105. The protrusions 111 extend outwards from an end portion of the tubing 102. The protrusions 105 and the protrusions 111 are configured to allow the tubing 102 to be inserted into the tubing accepting opening 109 but resist removal of the tubing 102 once inserted. In the exemplary connector 100, the protrusions 105 are inclined away from the opening 109 and the protrusions 111 are inclined away from a tubing opening 112 near the end portion of the tubing 102. Once the tubing 102 is pushed inside the opening 109 into the annular gap 107 and over the insert 101, the protrusions 105 and the protrusions 111 connect with each other such that the tubing 102 is locked inside the connector 100 and cannot be pulled out. In FIG. 3, the tubing 102 is shown inserted fully over the insert 101 such that the passage 106 extends through the tubing 102 and the protrusions 105 and the protrusions 111 are interlocked with each other. In an embodiment of the invention, the protrusions 105 are formed as a separate element that can be inserted into the annular gap 107. In another embodiment, the protrusions 111 are formed as a separate element that can be slipped over the end portion of the tubing 102 through the tube opening 112. The connector 100 also comprises an optional flange 108 to facilitate attachment of the connector 100 to a bioreactor, a cell bag, a manifold or other biotechnological equipment.

FIG. 4 shows an end view of the exemplary connector 100 as illustrated in FIG. 3. The connector 100 comprises the hollow insert 101 enclosing the passage 106 for fluid flow. The outer portion 103 comprises the protrusions 105 extending into the annular gap 107. The tubing 102 is shown to be inserted over the insert 101 such that the protrusions 111 are interlocked with the protrusions 105.

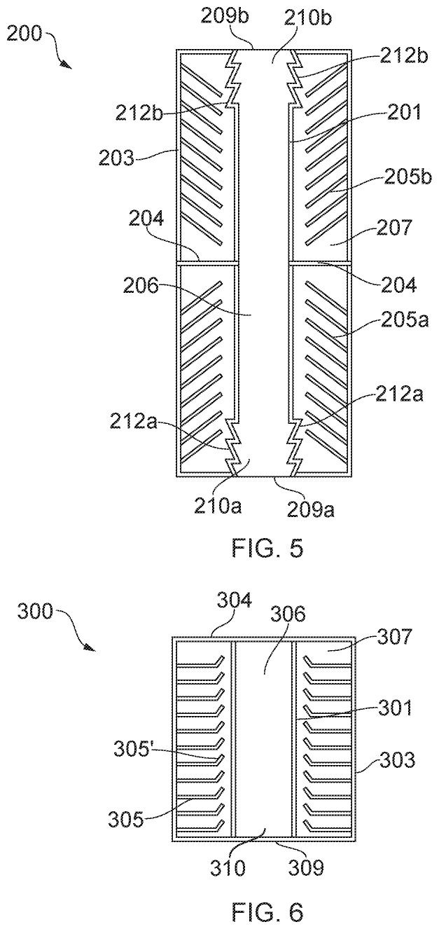

FIG. 5 shows a longitudinal sectional view of a two-way connector 200 according to a first embodiment of the invention. The two-way connector 200 can be connected to a first tubing and a second tubing. The connector 200 comprises a hollow insert 201 enclosing a passage 206. The passage 206 allows fluid flow from the first tubing to the second tubing through the connector 200 and vice versa. The insert 201 comprises a first end 210a and a second end 210b at opposite ends of the connector 200. In the connector 200, the ends 210a and 210b of the insert 201 have one or more barbs 212a and 212b to facilitate entry of the insert 201 inside the first tubing and the second tubing. The insert 201 is surrounded by an outer part 203 such that there is a generally annular gap 207 between the insert 201 and the outer part 203. The annular gap 207 forms a first tubing accepting opening 209a and a second tubing accepting opening 209b at or adjacent to the first end 210a and the second end 210b of the insert 201 respectively. A joining member 204 rigidly connects the insert 201 to the outer part 203 at a region between the first end 210a and the second end 210b of the insert 201. The outer part 203 comprises a plurality of resilient protrusions 205a and 205b extending into the annular gap 207. The protrusions 205a and 205b are inclined away from the first opening 209a and a second opening 209b respectively to allow the first tubing and the second tubing to be inserted into the openings 209a and 209b respectively but resist removal of the first tubing and the second tubing once inserted.

FIG. 6 shows a longitudinal sectional view of a connector 300 according to a second embodiment of the invention. The connector 300 comprises a hollow insert 301 enclosing a passage 306 to allow fluid flow through the insert 301. Proximal end 310 of the insert 301 is configured to fit inside a tubing. In this embodiment, the proximal end 310 of the insert 301 is of a blunt design. The insert 301 is surrounded by an outer part 303 such that there is a generally annular gap 307 between the insert 301 and the outer part 303. The annular gap 307 forms a tubing accepting opening 309 at or adjacent to the proximal end 310 of the insert 301. A joining member 304 rigidly connects the insert 301 to the outer part 303 at a region spaced from the proximal end 310 of the insert 301. This is achieved by manufacturing the connector 300 as a single integral molding made of plastics, metal or any other material. The outer part 303 comprises a plurality of resilient protrusions 305 extending into the annular gap 307. In this embodiment, the protrusions 305 have ends 305′. The ends 305′ are inclined away from the opening 309 to allow the tubing to be inserted into the tubing accepting opening 309 but resist removal of the tubing once inserted.

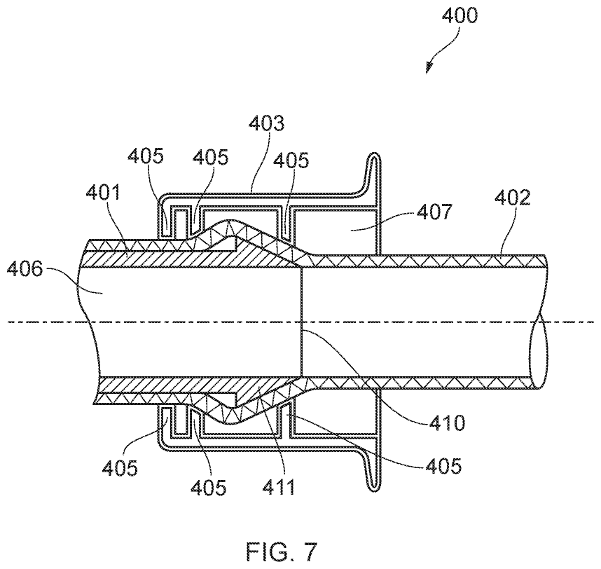

FIG. 7 shows a longitudinal sectional view of a connector 400 according to a third embodiment of the invention. The connector 400 comprises a hollow insert 401 with a barb 411 near a proximal end 410 of the insert 401 and a tubing locking ring 403 for clamping a tube 402 around the barb 411 of the insert 401. The barb 411 extends radially outwards from the insert 401. The locking ring 403 comprises an annular gap 407 extending along an axis, which is co-axial with the insert 401 in use. In FIG. 7, the insert 401 with the barb 411 is shown to be inserted in the tube 402. The locking ring 403 has three locking protrusions 405 respectively spaced along the axis and extending into the annular gap 407. The locking ring 403 is positioned over the tube 402 fitted over the insert 401 such that the tapered ends of two adjacent locking protrusions 405 engage the tube 402, one on each side of the barb 411. As shown in FIG. 7, the two adjacent locking protrusions on either side of the barb 411 have their innermost ends tapered to a point for gripping the tube 402. In another example of this embodiment, the locking ring 403 could have two, four, five or more protrusions.

In a method of the invention a leak-proof connection is made between a connector and a flexible tubing by pushing an insert of the connector inside an end of the tubing wherein the connector comprises a first hollow insert for allowing fluid flow, having a proximal end configured to fit inside the tubing, a first outer part, a joining member, rigidly connecting the first insert to the outer part at a region spaced from the proximal end of the first insert and a generally annular gap between the first insert and the first outer part, which forms a tubing accepting opening at or adjacent the proximal end of the insert; the first outer part further comprises plural resilient protrusions extending into the gap, allowing the tubing to be inserted, but resisting removal of the tubing once inserted and wherein at least the insert, the joining member and the outer part are formed as a single integral molding and wherein the pushing of the insert causes a seal to be formed between an internal face of the tubing and an outer diametral face of the insert.

The invention is not to be seen as limited by the embodiments described above, but can be varied within the scope of the appended claims as is readily apparent to the person skilled in the art. For instance, the invention would work equally well with an insert having features to enhance the friction fit to the tubing in which the insert is inserted, for example a barbed insert. Other embodiments of the invention include an angled connector and a connector for connecting a plurality of flexible tubes (multi-way connector). Other embodiments of the invention include a different design of the protrusions which similar to other embodiments allows the tubing to be inserted but not pulled out. the proximal end of the insert can be fully contained inside the proximal end of the outer part or the proximal end of the insert could be protruding outwards or flush with the proximal end of the outer part. The protrusions on the outer part of the connector could be integrally molded with the outer part, the insert and the rigid member connecting the outer part and the insert.

1. A connector for flexible tubing, the connector comprising:
a first hollow insert for allowing fluid flow, having a proximal end configured to fit inside the tubing;
a first outer part;
a joining member, rigidly connecting the first insert to the outer part at a region spaced from the proximal end of the first insert; and
a generally annular gap between the first insert and the first outer part, which forms a tubing accepting opening at or adjacent the proximal end of the insert;
the first outer part further comprising plural resilient protrusions extending into the gap, allowing the tubing to be inserted, but resisting removal of the tubing once inserted; and
wherein at least the insert, the joining member and the outer part are formed as a single integral molding.

2. The connector of claim 1, wherein the single integral molding is made of plastics or metal material.

3. The connector of claim 1, wherein the resilient protrusions are formed as an element separate from and insertable into the annular gap.

4. The connector of claim 1, wherein the hollow insert has at least one barb on its outer surface to facilitate insertion of the tubing over the said insert.

5. The connector of claim 1, wherein the hollow insert is configured to make a seal between an internal face of the tubing and an outer diametral face of the insert.

6. The connector of claim 1, wherein the connector further includes a second insert and a second outer part fluidically connected to the first insert and the first outer part to provide a further tube accepting opening, the second insert and the second outer part being rigidly connected also by the joining member, the connector thereby being configured to accept tubing at more than one fluidically connected openings and to allow fluid to flow through the first insert and into the second insert.

7. A connector and a flexible tubing assembly comprising:
a connector as claimed in claim 1;
tubing having one or more ends formed with protrusions complimentary to the protrusions of the first and/or second outer parts further configured to allow the tubing to be inserted over the connector but resist removal of the tubing once inserted.

8. The connector of claim 1, wherein the joining member forms part of any one of:
a bioreactor bag; a disposable fluid containment; a fluid manifold or a bioproces sing instrument.

9. The connector and the tubing assembly of claim 7, wherein said protrusions of the first and/or second outer part, and the protrusions formed on the tube, together are in the form of ratcheting teeth.

10. The connector of claim 1, wherein the protrusions of the first and/or the second outer part, are inclined away from the opening, or have ends which are so inclined.

11. A method of connecting a flexible tubing to a connector wherein the connector comprising:
a first hollow insert for allowing fluid flow, having a proximal end configured to fit inside the tubing;
a first outer part;
a joining member, rigidly connecting the first insert to the outer part at a region spaced from the proximal end of the first insert; and
a generally annular gap between the first insert and the first outer part, which forms a tubing accepting opening at or adjacent the proximal end of the insert;
the first outer part further comprising plural resilient protrusions extending into the gap, allowing the tubing to be inserted, but resisting removal of the tubing once inserted; and
wherein at least the insert, the joining member and the outer part are formed as a single integral molding;
wherein the method comprising:
pushing the insert of the connector inside an end of the tubing such the tubing is received in the tubing accepting opening of the connector wherein the pushing of the insert causes a seal to be formed between an internal face of the tubing and an outer diametral face of the insert.

12. A bioprocessing tubing locking ring for clamping a tube around a barb of a tube insert, the locking ring comprising an annular gap extending along an axis, which axis is intended to be generally co-axial with the insert in use and comprising a plurality of locking protrusions respectively spaced along the axis and extending into the annular gap for engaging the tube in use, wherein at least one of the locking protrusions has an innermost end which is tapered to a point for gripping the tube.

13. A bioprocessing tube holding assembly comprising a tube fitted over a barb of a tube insert, the insert comprising a proximal end for inserting into the tube, and a distal end which is connectable to or part of other bioprocessing apparatus, the insert further comprising a tube locking ring as claimed in claim 12, wherein a respective adjacent pair of the plurality of locking protrusions of the locking ring are together configured to clamp the tube onto the insert one on each side of the barb.
